(19) United States
(12) Patent Application Publication
(10) Pub. No.: US 2005/0176012 A1
(43) Pub. Date: Aug. 11, 2005

(54) Differential indication of labeled molecules

(76) Inventors: Wojciech Wozny, Nieder-Olm (DE); Michael Cahill, Loerzweiler (DE)

Correspondence Address: NATH & ASSOCIATES, 1030 15th STREET, NW, 6TH FLOOR, WASHINGTON, DC, 20005, US

(21) Appl. No.: 10/516,392

(22) PCT Filed: Jun. 3, 2003
(86) PCT No.: PCT/EP03/05797

(30) Foreign Application Priority Data
Jun. 3, 2002 (DE) 102 25 841.4

(51) Int. Cl. C12Q 1/68; G06F 19/00; G01N 33/48; G01N 33/50; G06K 9/00

(52) U.S. Cl. 435/6; 382/128; 702/20

(57) ABSTRACT

The invention relates to a method for determining the local distribution of at least two sets of point radiation objects each with a set-specific radiation type on a common support surface.

### APPLICATION FIELD AND PRIOR ART

The invention relates to a method for determining the local distribution of at least two sets of point radiation objects each with a set-specific radiation type on a common support surface.

One important application of proteomic studies is the determination of the difference in the frequencies of particular proteins or protein isoforms between two or more regimes of experimental interest, for example the frequency of proteins from control cells, tissues or organisms, in comparison with the corresponding frequency from pharmacologically treated cells, tissues or organisms.

The protein intensities for protein spots of different gels from different experimental regimes in the study are typically compared in order to determine whether the amount of protein in these protein spots differs between the experimental regimes. This is a differential representation principle which is also referred to as multicolor analysis. The allocation of protein spot coordinates and the identification of corresponding protein spots on various 2D gels is referred to as a matching procedure, and is potentially problematic owing to the irreproducible behavior of different gels which are produced by 2D PAGE. The matching procedure is therefore a time-consuming step, and at present it can presently be automated only to a minor extent.

For example, a rough multicolor analysis can be carried out by measuring the various samples in different experimental systems. In this case, for example, proteins from different samples are treated in respectively separate 2D PAGE experiments, followed by detection of the spots which correspond to proteins in the various 2D PAGE gels, and comparison of the spot patterns obtained for each sample of each gel or each group of replicated gels. The spot patterns are typically stored in graphical gel image files, for example TIFF files. Conventional software program algorithms align these gel image files with one another, so that pixels in all the images can be compared with pixels from other images. A “composite gel image” of replicated 2D PAGE gel images is typically produced for a sample, which contains averages of the spot intensities of all the replicate gels for a particular sample. The composite gels of different samples are typically compared with one another in order to reveal differences in protein frequencies between the samples. The two major restrictions of this approach are spot matching, which makes possible the correct allocation of the corresponding spots, and quantitative evaluation of the spots, which allows comparison of the protein amounts in spots of different 2D gels.

In order to create a more refined differential representation, analysate molecules of different samples are typically labeled with reagents or modified so that the samples can be mixed and analyzed together. The elaborate and error-prone matching procedure can be avoided in this way. Because of their labeling, the proteins of each sample can subsequently be detected irrespective of the presence of the proteins from the other sample(s).

The DIGE fluorescent reagents based on Cy dyes, which are available from Amersham Biosciences (Freiburg, Germany), are one example. Proteins from the two samples are labeled separately with different fluorescent reagents, then mixed and subjected to the 2D PAGE electrophoresis together. The various fluorescent groups in each sample are excited by different wavelengths, and emit light at different wavelengths. By using suitable light filters, it is possible to obtain images of proteins in each sample irrespective of the distribution of the proteins from the other sample. These images can then be analyzed by suitable software packages.

Another approach for a more refined differential representation involves labeling analysate molecules from different samples with radioactively radiating isotopes. The radioactive labeling of analysate molecules and their detection by suitable detectors is several orders of magnitude more sensitive than other methods, for example the aforementioned method using Cy dyes.

An analysis method in which analysate molecules are labeled with radioactively radiating isotopes is described in:

Monribot-Espagne C, Boucherie H, Differential gel exposure, a new methodology for the two-dimensional comparison of protein samples. Proteomics, 2002, 2:229-240.

When analysate molecules are labeled with radioactively radiating isotopes, it is necessary to determine the radioactive radiation or the activity of the isotopes. The nuclei of radioactive isotopes are unstable. They tend to decay spontaneously by releasing particles or photons. Several different types of particles are released by radioactive substances. The electron, the positron, the alpha particle and the neutron are generally the important ones. The emission of these particles is often, although not always, accompanied by the radiation of gamma rays. Another type of radioactive decay is the spontaneous capture of a K shell electron by the nucleus, which is known as K electron capture. Radiation detectors are devices which have been developed in order to detect these various types of radiation.

The following spatially resolving detectors are used for the quantitative analysis of radioactive molecules which are present in an array or on a surface, such as a DNA array or a 2D PAGE protein gel, and they replace a photographic film for this purpose. Out of the four examples cited, detector types 2, 3 and 4 are based on the above principles.

1. Autoradiography with phosphor imagers, which operate according to the principles of devices that use an image plate, such as the devices currently available from Fuji Photo Film Co. Ltd (Tokyo), Packard Bioscience GmbH (Dreieich, Germany), AGFA-Gevaert N. V. (Mortsel, Belgium) or Amersham Biosciences (Freiburg, Germany). Amemiya Y, Miyahara J. Imaging plate illuminates many fields. Nature, 1998, 336:89-90.

Miyahara J. The imaging plate: a new radiation image sensor. Chemistry Today, 1989, 223:29-36.

2. PPAC (parallel plate avalanche chamber) ionization gas chambers which are coupled to microchannel plate analyzers in order to detect beta particles, such as the beta imager which is marketed by BioSpace Measures (Paris), and a similar device which is marketed by Packard Bioscience (Dreieich, Germany).

In these imagers, light from the PPAC is focused by a lens into a multifiber image intensifier tube, the signal from which is recorded by a CCD camera.

Laniece P, Charon Y, Dumas S, Mastrippolito R, Pinot L, Tricoire H, Valentin L. HRR1: a high resolution radioimager for fast, direct quantification in in situ hybridization experiments. Biotechniques, 1994, 17:338-345.

Crumeyrolle-Arias M, Latouche J, Laniece P, Charon Y, Tricoire H, Valentin L, Roux P, Mirambeau G, Leblanc P, Fillion G et al. “In situ” characterization of GnRH receptors: use of two radioimagers and comparison with quantitative autoradiography. J. Recept Res, 1994, 14:251-265.

Jeavons A G. Method and apparatus for quantitative autoradiography analysis. 11 Aug. 1992. U.S. Pat. No. 5,138,168. Proprietor: Oxford Position Systems Limited.

Decristoforo C, Zaknun J, Kohler B, Oberladstaetter M, Riccabona G. The use of electronic autoradiography in radiopharmacy. Nucl. Med. Biol., 1997, 24:361-365.

3. Devices such as the Micro Imager (BioSpace Measures, Paris) in which a scintillation surface for the detection of beta, gamma and alpha particles is coupled with microchannel plate analyzer devices. In these imagers, light from the scintillation surface enters a multifiber image intensifier tube, the signal from which is acquired by a CCD camera.

Laniece P, Charon Y, Dumas S, Mastrippolito R, Pinot L, Tricoire H, Valetin L. HRR1: a high resolution radioimager for fast, direct quantification in in situ hybridization experiements. Bio-techniques, 1994, 17; 338-345.

Crumeyrolle-Arias M, Latouche J, Laniece P, Charon Y, Tricoire H, Valentin L, Roux P, Mirambeau G, Leblanc P, Fillion G et al. “In situ” characterization of GnRH receptors: use of two radioimagers and comparison with quantitative autoradiography. J. Recept Res, 1994, 14:251-265.

Jeavons AG. Method and apparatus for quantitative autoradiography analysis. 11 Aug. 1992. U.S. Pat. No. 5,138,168. Proprietor: Oxford Position Systems Limited.

Decristoforo C, Zaknun J, Kohler B, Oberladstaetter M, Riccabona G. The use of electronic autoradiography in radiopharmacy. Nucl. Med. Biol., 1997, 24:361-365.

4. Scintillation crystal array imagers, which use spatially resolving photomultipliers such as the MPD (Multi Photon Detection) imager marketed by BioTraces Inc. (Herndon Va., USA) and ProtoSys AG (Mainz, Germany).

Drukier A J, Sagdejev I R, Ultralow background multiple photon detector. 2 Feb. 1999. U.S. Pat. No. 5,866,907. Proprietor: BioTraces Inc. (Herndon Va.).

In contrast to the above detector types 2, 3 and 4, a phosphor imager imaging plate (phosphor imager IP) is a film-like radiation image sensor which contains a specially designed phosphor that absorbs and stores the radiation energy so that it can be released again at a later time and measured in a suitable phosphor imager readout device. A phosphor imager device is a combined system consisting of a phosphor imager readout device and a phosphor imager imaging plate, and will be referred to below as a phosphor imager. A phosphor is a powder or a crystalline substance which emits light after being affected by photons with particular properties or chemical reactions. Light emission (luminescence) by a phosphor may be instantaneous (fluorescence), delayed (phosphorescence) or photostimulated luminescence (PSL). The phosphor of the phosphor imager IP uses the PSL phenomenon, which is neither fluorescence nor phosphorescence. PSL uses a substance which stores the energy of an original stimulation, for example the energy of a photon that is emitted by a radioactively decaying atom, in the electron orbitals of atoms of the phosphor. This energy is emitted as light when the phosphor is stimulated by light with a wavelength longer than that of the first stimulation. In this way, the phosphor stores information about the amount of radiation to which it was exposed, and outputs this information as light which can be quantitatively analyzed in a suitable device, such as a commercially available phosphor imager.

The irradiation of samples in the phosphor imager is carried out in a similar same way as the exposure of a photographic film. The irradiated phosphor imager IP is scanned with a focused laser beam (for example a He—Ne laser) during the transfer. The PSL released when the phosphor crystals are irradiated with the laser light is collected in a photomultiplier tube (PMT) by a light collection conductor and converted into electrical signals.

Among the types of radioactive measuring devices as discussed above (phosphor imagers, PPAC ionization gas chamber detectors, scintillation crystal array imagers with a spatially resolving photon multiplier, scintillation surfaces imaged by a CCD camera), scintillation crystal array imagers can differentiate various isotopes by using energy and particle type (beta/gamma) and analyzing the detected pulse shape of different emitted particles. PPAC ionization gas chamber detectors (for example beta imagers) and scintillation surfaces imaged by a CCD camera (for example microimagers) require detection of the extremely weak beta particles of H-3 in addition to another isotope in order to distinguish different radioisotopes, as indicated by the manufacturer in their description. These detectors cannot detect H-3 labeled proteins directly from a 2D PAGE gel, because the polyacrylamide matrix absorbs the weak H-3 beta particles. Analysate molecules must be removed from the polyacrylamide gel, for example by blotting onto a membrane, so that they can be detected by PPAC ionization gas chamber detectors. This is time-consuming and works only with a variable efficiency, for example for proteins with a differing molecular weight. Analysate molecules labeled with more strongly beta-emitting isotopes, such as S-35, can be made visible efficiently by ionization gas chambers even when they are still in a polyacrylamide gel matrix, and they do not need to be blotted. However, scintillation crystal array imagers which use a spatially resolving photon multiplier and ionization gas chambers occupy a device throughout the entire radioactive irradiation. Analyses which require a high throughput, for example in protein or genome studies, are therefore very expensive.

Further conventional differential detection methods will be described below, although these cannot be used for differential detection of analysate molecules on a support surface.

The quantitative determination of the concentration of two different elements present in a material sample by using two measurements with one detector is described in: Sastri et al., Anal. Chem., 1981, 53:765-770.

Only the concentration of the elements in a material sample is calculated in this method, however, rather than their local distribution on the sample surface, for example. The method described here is therefore unsuitable for the differential analysis of analysate molecules which are labeled with different isotopes.

The hyperspectral processing software MultiSpec© from Purdue University (Purdue Research Foundation, West Lafayette, Ind.) uses spectral measurements of images in order to extract or identify different features of satellite images. The MultiSpec© concept involves decomposing the spectral occupancy of pixels of an image into a series of images, all of which are derived from a master image for which the primary data were all recorded at the same time. In this method, starting from a master image, various images derived from this original image are thus produced by mathematical methods.

David Landgrebe, Information Extraction Principles and Methods for Multispectral and Hyperspectral Image Data, Chapter 1 of Information Processing for Remote Sensing, edited by C. H. Chen, published by World Scientific Publishing Co., Inc, 1060 Main Street, River Edge, N.J. 07661, USA, 2000.

Since the radioactive labeling of analysate molecules is often the method of choice for achieving the requisite analytical performance in many studies, for example biological studies, and since the differential detection of analysate molecules from two or more samples in one analysis is desirable, there is a great need to carry out such differential detection with radioactively labeled analysates or other molecules.

Object and Solution

It is therefore an object of the invention to provide a method which allows differential analyses, for example in protein or genome studies, with a high analytical performance and a high throughput together with comparatively low costs, and which helps to overcome the described disadvantages of the prior art.

This object is achieved by a method having the features of claim 1. Preferred refinements of the invention are set out in dependent claims 2 to 19. The wording of all the claims is hereby included by reference in the content of this description by reference.

In the method according to the invention, the local distribution of at least two sets of point radiation objects each with a set-specific radiation type on a common support surface is determined by the following steps: a) finding a first local distribution of the radiation intensity of the support surface, b) modifying the radiation intensity of at least one set-specific radiation type with an associated modification factor, c) finding at least a second local distribution of the radiation intensity of the support surface, and d) calculating the local distributions of each of the at least two sets of point radiation objects individually from the first and the at least second local distributions which have been found.

The local distribution of point radiation objects means, for example, the radiation intensity and/or the number of the respective point radiation objects per unit area over the position or over the support surface to be studied. The local distributions of at least two sets of point radiation objects, each with a set-specific radiation type, are studied. A point radiation object is an object, for example an atom or its isotope and/or a molecule consisting of groups of atoms or isotopes, which emits its specific radiation uniformly or at least theoretically uniformly to all sides, that is to say over the solid angle. The set-specific radiation type may, for example, be alpha, beta, gamma and/or X-radiation and/or light with a specific wavelength. The at least two sets respectively consist of point radiation objects with the same set-specific radiation type and lie together on the substrate surface. The substrate surface is an substantially flat structure, for example a 2D PAGE gel.

A first local distribution of the radiation intensity, that is to say the radiation power per unit area, of the support surface is found in the first step a) of the method according to the invention.

The radiation intensity of at least one set-specific radiation type is modified with an associated modification factor, which is known or determined or can be calculated, in the second step b). The modification may be a reduction or an increase in the intensity of the set-specific radiation type. The modification factor is given in %, 0% meaning complete suppression of the radiation intensity, 100% constancy of the radiation intensity and values greater than 100% an increase in the radiation intensity.

It is possible to modify all the intensities of the set-specific radiation types, only some of the intensities of the radiation types or only one intensity of a radiation type by an associated modification factor. The ratio or ratios of the modification factors of the set-specific radiation types preferably has or have a value or values not equal to 1, that is to say the modification factors of the set-specific radiation types preferably differ from one another.

At least a second local distribution of the radiation intensity of the support surface is found in a further step c) after the modification of the radiation intensities.

The local distributions of each of the at least two sets of point radiation objects are calculated individually in a fourth step d) from the first and the at least second local distributions which have been found.

On the basis of the at least two local distributions which have been found, the local distribution of each set of point radiation objects on the support surface is determined separately by the calculation, irrespective of the distribution of the other sets of point radiation objects on the support surface. Separation into the set-specific local distributions is not carried out directly by the detector, but only by calculation on the basis of the local distributions which have been found, which represent a superposition of the detected signals of the individual radiation sources.

In an advantageous refinement of the invention, the modification step b) and the step c) of finding the at least second local distribution of the radiation intensity of the support surface are repeated at least once with a different modification factor. This is necessary when more than two sets of point radiation objects are to be analyzed, since at least one local distribution with an associated modification of the intensity of the set-specific radiation type must be found in order to determine the individual local distribution of a set.

If only two sets of point radiation objects are being analyzed, the accuracy of the method can be improved by such a repetition. This is achieved, in particular, by obtaining the modification factors with different mechanisms, for example with the aid of an absorber and/or subsequently by radioactive decay.

In another advantageous refinement of the invention, a local distribution of the radiation intensity which has been found is represented by an associated pixel matrix, with a pixel value of the matrix representing the radiation intensity of an associated position on the support surface.

When finding the local distributions of the radiation intensity of the support surface, for example using a suitable position-resolving detector, the local distribution of the radiation intensity of the support surface is converted into a matrix arrangement of pixel values, in which case the pixel values may be proportional to the radiation intensity found by the detector. Each pixel is allocated a defined region of the surface.

In another advantageous refinement of the invention, the local distribution of each of the at least two sets of point radiation objects is determined individually on the basis of the pixel matrices.

In a purely illustrative example, the calculation may be carried out as follows when analyzing two sets A and B of point radiation objects and with a modification factor of 0%, that is to say complete suppression, for the radiation type of set A a modification factor of 100%, that is to say constancy, for the radiation type of set B.

Owing to the complete suppression of the radiation type A in the second local distribution which has been found, the superposition-free local distribution for the radiation type B is obtained directly. By subtracting the second local distribution which has been found, in which there are only signal components of the radiation type of set B, from the first local distribution which has been found, in which there are both of the signal components, the local distribution of the radiation type of set A is obtained without signal components of the radiation type B.

Said method allows a multicolor measurement by using the same detector or a similar detector, that is to say it allows a separate superposition-free representation of the local distribution of the individual point radiation sources. Conventional methods require the original monochromatic images of analysate molecules of each sample for a multicolor representation. The monochromatic images of each isotope are never measured per se in the present invention, but are calculated from the at least two local distributions or images which have been found.

In practice, the signal components attributable to the different radiation types are preferably modified or reduced by a factor of less than 100% or 95%. One possible way of calculating the superposition-free local distribution of the two sets of point radiation objects will be described below.

The following applies for an exemplary pixel value of the pixel matrix of the first local distribution which has been found:
A+B=X (1)

The following applies for the pixel value of the second local distribution which has been found:
MA+NB=Y (2):

Here
A=the signal contribution of radiation type A in the first local distribution which has been found
B=the signal contribution of radiation type B in the first local distribution which has been found
X=pixel value corresponding to the radiation intensity in the first local distribution which has been found
Y=pixel value corresponding to the radiation intensity in the second local distribution which has been found
M=modification factor for the set-specific radiation type A
N=modification factor for the set-specific radiation type B.
A and B are unknown and to be found.
X and Y are the measured intensities.

M and N can be determined by calibration or calculation. The ratio of the modification factors of two radiation types is defined as the ratio of the modification factor of the radiation type with the smaller modification factor and that of the radiation type with the larger modification factor (M/N with N>M). The ratios of the modification factors are defined in a corresponding way when there are more than two radiation types.

The values for A and B can be calculated by solving the equation system consisting of equations (1) and (2). This calculation can be carried out in a similar way for all pixels of the pixel matrix.

If the radiation intensity or the signal contribution of a radiation type at a position is known, it is thus also possible to calculate the number of point radiation objects per unit area at the position, since the radiation intensity and the number of point radiation objects per unit area are proportional to each other.

In a more general case with a number P of sets of point radiation objects, for which it is necessary to calculate P local distributions, the problem can be formulated by using a matrix representation:
F*X=I
where:
X=a vector with P elements, consisting of the signal contributions of the radiation types of the P sets of point radiation objects in the first local distribution which has been found
I=a vector with P elements, consisting of the pixel values of the P local distributions which have been found
F=a matrix with P*P elements, consisting of the P*P modification factors which have been calculated or determined for the P local distributions found for the P different sets of point radiation objects.

The individual signal contributions of the radiation types of the vector X can be calculated by inverting the matrix F according to the following equation:
X=F⁻¹*I

In a refinement of the invention, the local distribution of each of the at least two sets of point radiation objects is determined individually on the basis of intensities which are obtained by adding up pixel values of defined, in particular neighboring, elements of the pixel matrix. Instead of using the algorithm as described above for a pixel-by-pixel analysis, it is also possible to use the summed pixel intensities of particular regions which can be computed for the first and second local distributions that have been found. This is particularly recommendable for very noisy signals or in cases where the spatial distribution of the signals on the detector is variable for the different sets of point radiation objects.

Preferably, more measurements of the local distributions are carried out under modified conditions than would be necessary as a minimum. In this case, an optimization process is used in order to determine the individual local distributions of the point radiation objects. The accuracy of the method can be improved in this way.

In a refinement of the invention, a set of point radiation objects consists of at least one radiating, in particular radioactively radiating, type of isotope. For example, I-125 and/or I-131 may be used as isotopes.

In a refinement of the invention, a set of point radiation objects consists of light-emitting, in particular fluorescent, phosphorescent and/or luminescent, substances.

In a refinement of the invention, there is at least one calibration point with known radiation for at least one set of different point radiation objects at at least at one position on the support surface. The calibration is used to determine or calculate the respective modification factors, which are needed in order to calculate the individual local distributions of interest.

A calibration point may be an individual signal source, for example an individual radioactive isotope. The calibration is used for the purpose of calculating the absolute or relative percentage of the modification or reduction of signal intensities, which are due to the radiation sources or isotopes being studied.

The calibration may be carried out by incorporating a radioactive starting substance for one or more of the isotopes being studied in the measurement. The measured signal of this calibrating substance should preferably contain no or negligible components of all other radioactive sources apart from the calibrating substance. The specific activity of the calibrating substance before the measurement is preferably known. The calibrating substance is preferably available for all isotopes being studied. A calibration is also possible by using one or more radioactive substances which, located spatially together, contain known amounts of all the isotopes to be measured. In this case, the specific activities or radiation intensities of each isotope in the calibrating substance must be known. With the aid of suitable algorithms, on the basis of a plurality of local distributions which have been found, it is possible to calculate the contribution of each isotope to the respective pixel value of the local distribution, with one or more radiation intensities of the isotopes in the signal being modified in a local distribution. Other factors must of course be known in the latter case, for example the differential degree of the radioactive decay, etc.

When an absorber is used, the modification factors depend on the absorber material, its thickness and its mass absorption coefficient for the types of radioactive particles being absorbed. When differential images are being produced by virtue of the fact that both isotopes decay, the modification factors can be determined by measuring the relative intensities of the calibrating substances for the two isotopes. Alternatively, they may be calculated by using information relating to the exact time and duration of each irradiation and the decay rate constants for each radioisotope.

In a refinement of the invention, the ratio of the modification factors of the set-specific radiation types is between 5% and 90%, preferably between 10% and 70%, in particular between 15% and 50%. The possibility of working with modification factors that do not fully suppress one radiation type significantly increases the number of radiation sources or detectors which can be used. In particular when the modification factors are obtained by radioactive decay of differently radiating isotopes, it is possible to calculate the separate local distributions of the isotopes even without complete decay of one isotope type.

In a refinement of the invention, the modification factor is obtained by using at least one absorber. An absorber reduces the intensity of the radiation (particles or photons) passing through the material. The absorber may consist of metal, metal alloy, plastic, polymethyl methacrylate, polytetrafluoroethylene and/or another suitable material. It is essential for the function of the invention that the emitted radiation types which generate the signal on the detector, for example photons from I-125 isotopes and beta particles from I-131 isotopes, be be absorbed or attenuated with different modification factors or reduction factors by the absorber. Absorbers made of elements with a low atomic number are more effective for this purpose, for example aluminum foil or thin polymethyl methacrylate, since these absorbers absorb beta particles more strongly than photons. Absorbers made of a very thin layer of elements with a high atomic number may furthermore be used, since these absorbers absorb photons more strongly than beta particles. The absorber thickness plays a key role in the absorption efficiency.

In a refinement of the invention, the modification factor is obtained by radioactive decay of the respective point radiation objects of the at least two sets, with the respective point radiation objects of the at least two sets preferably having different half-lives. The modification or reduction of the radiation intensity is not due to an absorber in this case, but to a difference in the decay rates of the point radiation objects. The isotopes I-125 and I-135, for example, are well suited to this purpose since their half-lives differ by a factor of almost 7.5. Nevertheless, other isotopes may also be used for the invention.

In a refinement of the invention, the modification factor is obtained by a differing sensitivity of a detector, used for finding a local distribution of the radiation intensity of the support surface, for the respective set-specific radiation type. The modification factors of the radiation intensities of the set-specific radiation types are obtained in this case by the differing sensitivities of the detectors being used to the radiation emitted by the at least two sets of point radiation objects. The differing sensitivities are preferably selected so that the ratio of the modification factors is preferably more than 5%.

In a refinement of the invention, the local distributions of the radiation intensity are found by using a position-resolving detector for alpha, beta, gamma and/or X-radiation.

In a refinement of the invention, the local distributions of the radiation intensity are found by using a so-called phosphor imager detector. Such a phosphor imager detector is also referred to as a phosphor imager imaging plate or phosphor imager IP. The phosphor imager detector preferably operates on the basis of photostimulated luminescence. A differing sensitivity of a phosphor imager detector, used for finding a local distribution of the radiation intensity of the support surface, for the respective set-specific radiation type is preferably achieved by the phosphor of the phosphor imager detector additionally containing atoms with a high atomic number Z, in particular lead which, for example, may be contained in a lead compound, and/or atoms with a low atomic number Z. When atoms with a high or low atomic number Z are incorporated into the phosphor imager detector during manufacture of the phosphor imager detector, its detection sensitivity is respectively increased or decreased for gamma radiation. This means that the ratio of the sensitivities for different radiation types changes. In this case, additional atoms are incorporated which are not necessary for the light storage in the phosphor imager detector, but which modify the absorption efficiency of the phosphor imager detector. In this way, it is possible to discriminate between alpha, beta, gamma and neutron radiation and radiation with a differing energy. In particular, the ratio of the sensitivity for gamma radiation to that for beta radiation, in particular for beta radiation with energies higher than 20 keV which can pass through the phosphor cladding of the detector, can be modified by:
1) incorporating elements with a high atomic number Z,
2) incorporating elements with a low atomic number Z.

In a refinement, the first local distribution of the radiation intensity of the support surface is found by using a first phosphor imager detector, and the at least second local distribution of the radiation intensity of the support surface is found simultaneously by using at least a second phosphor imager detector, the sensitivity of the first phosphor imager detector and the sensitivity of the at least second phosphor imager detector differing for the respective set-specific radiation type. This means that at least two phosphor imager detectors or phosphor imager IPs, which have a different ratio of the sensitivities to the radiation of the different point radiation objects, are used during the measurement. This makes it possible to measure or find the respective radiation intensities in quick succession or simultaneously. The differing sensitivities of the different IP materials make it possible to calculate the local distribution of the respective point radiation objects, even if there has not been a sufficient difference due to the decay of the isotopes being used. This procedure may of course also be combined with a waiting time between the measurements, so as to utilize decay of the isotopes as well.

In a refinement of the invention, the first and the at least second phosphor imager detectors are applied to opposite sides of the support surface. In this way, two measurements can be carried out synchronously or simultaneously on the front and rear sides of the samples or support surfaces to be measured, by using phosphor imager IPs with a differing sensitivity. The differing sensitivities of the different IP materials make it possible to calculate the local distribution of the point radiation objects from this.

In a refinement of the invention, the local distributions of the radiation intensity are found by using a so-called flat panel detector. Such flat panel detectors make it possible to fabricate real-time digital X-ray imagers. They convert X-rays directly into a matrix of digitally encoded pixels so that the detection efficiency and the evaluation speed are increased.

If the modification factor is to be obtained by a differing sensitivity of a detector, used for finding a local distribution of the radiation intensity of the support surface, for the respective set-specific radiation type, for example, then a phosphor imager detector and a flat panel detector may be used, for example, in order to carry out a respective measurement of the local distribution of the radiation intensity of the support surface. The local distribution of the point radiation objects can be calculated individually from the local distributions measured using the different detectors.

In a refinement of the invention, the first and the at least second local distributions of the radiation intensity are found by using the same type of detector. In principle, one detector does not allow clear discrimination between different radiation types, for example beta particles or photons which, for example, come from two different isotopes, or radiation from isotopes with different half-lives. A multicolor measurement, that is to say a separate superposition-free measurement of the local distribution of the individual types of point radiation sources, is possible with the aid of said method by using the same detector or a similar detector. Here, similar detectors are detectors of the same type or of the same design which, for example, do not differ or differ only insubstantially in terms of their sensitivity to specific radiation types. This simplifies the structure of an analysis device and therefore saves on costs.

In a refinement of the invention, the at least two substances to be analyzed are respectively labeled with a set of point radiation objects, the substances labeled in this way are mixed and the mixture is subsequently applied to the support surface, particularly in the form of a flat analysis medium. The substances to be analyzed may preferably be peptides, proteins and/or oligonucleotides with, for example, a first and a second protein sample which may respectively consist of a multiplicity of different proteins being labeled with respectively different isotopes. After labeling, the proteins of the two samples are mixed and applied together to an analysis medium, in particular a protein gel, nucleic acid array, protein array, ELISA array and/or a blot. A common separation of the substances or proteins to be analyzed in the different samples is subsequently carried out on the analysis medium with the aid of specific properties which, for example, are determined by the analysis medium. Following the separation, the local distribution of the point radiation objects is then calculated individually for each set of point radiation objects by the method according to the invention. Using the local distribution of the specific point radiation objects, it is then possible for the local distribution of the proteins to be studied in the respective samples to be deduced separately from each other.

### BRIEF DESCRIPTION OF THE DRAWINGS

Advantageous embodiments of the invention described below are represented in the drawings in which:

FIG. 1A shows the local distribution, found using a phosphor imager, of the radiation intensity of a mixture of 2 protein samples separated with the aid of a 2D gel, one sample having been labeled with isotopes of the I-125 type and the other sample having been labeled with isotopes of the I-131 type,

FIG. 1B shows the local distribution, found using the phosphor imager, of the radiation intensity of the 2D gel in FIG. 1A after a waiting time of 82 days,

FIG. 1c shows the local distribution, calculated with the aid of the method according to the invention, of the radiation intensity of the protein sample which was labeled with isotopes of the I-125 type,

FIG. 1D shows the local distribution, calculated with the aid of the method according to the invention, of the radiation intensity of the protein sample which was labeled with isotopes of the I-131 type,

FIG. 1E shows the difference of the local distributions in FIG. 1C and FIG. 1D, the protein sample which was labeled with isotopes of the I-125 type being represented in blue, and the protein sample which was labeled with isotopes of the I-131 type being represented in orange,

FIG. 1F shows the local distribution, found using the phosphor imager, of the radiation intensity of the 2D gel in FIG. 1A after a waiting time of 23 days,

FIG. 1G shows the local distribution, calculated with the aid of the method according to the invention, of the radiation intensity of the protein sample which was labeled with isotopes of the I-125 type, the local distribution in FIG. 1F having being used for the calculation as the second local distribution which was found,

FIG. 1H shows the local distribution, calculated with the aid of the method according to the invention, of the radiation intensity of the protein sample which was labeled with isotopes of the I-131 type, the local distribution in FIG. 1F having being used for the calculation as the second local distribution which was found,

FIG. 1I shows the difference of the local distributions in FIG. 1G and FIG. 1H, the protein sample which was labeled with isotopes of the I-125 type being represented in blue, and the protein sample which was labeled with isotopes of the I-131 type being represented in orange,

FIG. 2A shows the local distribution, found using a phosphor imager, of the radiation intensity of protein samples, labeled with isotopes of the I-125 type and isotopes of the I-131 type, of bovine serum albumin in different dilution stages and mixing ratios without using an absorber,

FIG. 2B shows the local distribution, found using the phosphor imager, of the radiation intensity in FIG. 2A while using an absorber,

FIG. 2C shows the local distribution, calculated with the aid of the method according to the invention, of the radiation intensity of the isotopes of the I-125 type,

FIG. 2D shows the local distribution, calculated with the aid of the method according to the invention, of the radiation intensity of the isotopes of the I-131 type,

FIG. 2E shows the difference of the local distributions in FIG. 2C and FIG. 2D, the isotopes of the I-125 type being represented in blue and the isotopes of the I-131 type being represented in orange.

### FIRST EMBODIMENT

In a first embodiment of the invention, two protein samples, each of which consists of a multiplicity of different proteins, are labeled with respectively different isotopes of the element iodine. The first sample is labeled with I-125 and the second sample with I-131. In order to calibrate the mass scale, special mass reference proteins are labeled with I-125. I-125 has a half-life of 60.14 days and decays by electron capture, resulting in the production of X-rays and gamma rays in the range of from 27 keV to 35 keV. I-131 has a half-life of 8.06 days and decays by the various mechanisms of beta emission.

The samples are subsequently mixed and separated with the aid of a 2D gel. In order to determine the modification factors, calibration points for the two isotope types are applied to the left-hand side the gel. They are used to calculate the modification factors which are needed for calculating the individual local distributions.

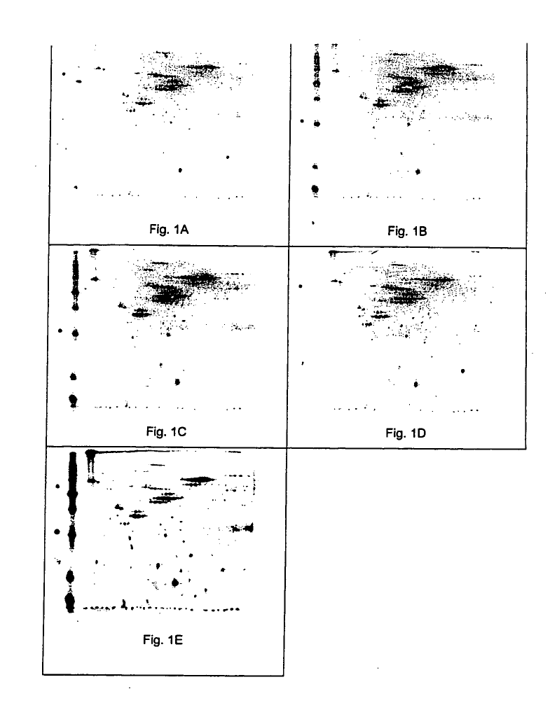

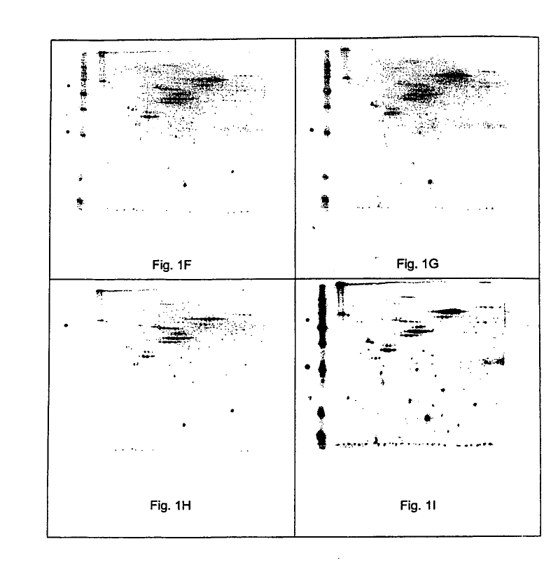

After the 2-dimensional separation, the phosphor imager IP is exposed for a predetermined time to the sample or the radiation from the sample. The phosphor imager IP is sensitive to both low-energy X-rays, gamma rays and beta particles. The relatively energetic photons from the I-131 decay are absorbed or detected with only a low efficiency by a phosphor imager IP. The phosphor imager IP integrates the radiation intensities of the two radiation sources. The local distribution of the stored radiation energy of the phosphor imager IP is subsequently read by a reader and formed into a pixel matrix or image, which is shown in FIG. 1A. The local distribution or pixel matrix found in this way contains components which come from both I-125 and I-131. After a waiting time of 82 days, the local distribution of the sample is determined again as described above. This local distribution is shown in FIG. 1B. Owing to the different half-lives of the two isotopes, the radiation intensity or activity of 1-125 has decreased by a factor of about two. In contrast to this, the radiation intensity of the I-131 remaining on the gel is less than 1% of the original radiation intensity. FIG. 1B therefore contains almost exclusively signal components from I-125.

The differing modification of the radiation intensities in this embodiment is due only to the inherent properties of the different radioactive decay of the two isotopes, rather than to an absorber.

Subsequently, the local distribution of the individual isotopes is calculated separately with the aid of the method according to the invention. The intensity over a region of 4×4 neighboring pixels was employed for the calculation, i.e. added up, with a Gaussian filter being used. The mutual alignment of the local distributions which have been found is carried out with the aid of corresponding reference points, which are respectively provided at the same positions of the local distribution. FIG. 1C shows the calculated local distribution of the radiation intensity or isotope amount of the protein sample which was labeled with isotopes of the I-125 type, and FIG. 1D shows the calculated local distribution of the radiation intensity of the protein sample which was labeled with isotopes of the I-131 type. FIG. 1E shows the difference of the local distributions in FIG. 1C and FIG. 1D, the protein sample which was labeled with isotopes of the I-125 type being represented in blue and the protein sample which was labeled with isotopes of the I-131 type being represented in orange. Black means that there is an equal number of the two isotopes.

Of course, the local distribution of the radiation intensity may also be determined without a position-resolving detector. For this purpose, the support surface to be studied is divided into smaller pieces by suitable separation, for example cutting, and the radiation intensity of these pieces is determined using a conventional “single-source” detector. For example, the radiation type and the radiation intensity may be determined by using gamma and/or beta spectroscopy.

### SECOND EMBODIMENT

In a second embodiment of the invention, with the same procedure as in the first embodiment, the waiting time between finding the first and second local distributions is reduced from 82 days to 23 days. FIG. 1F shows the local distribution, found using the phosphor imager, of the radiation intensity of the 2D gel in FIG. 1A after a waiting time of 23 days. Owing to the not yet complete decay of 1-131, both isotopes now contribute to the local distribution which has been found. The amplification factors are therefore substantially more than 0% for both isotopes. Likewise, the ratio of the modification factors is substantially more than 0%.

FIG. 1G shows the local distribution, calculated with the aid of the method according to the invention, of the radiation intensity of the protein sample which was labeled with isotopes of the I-125 type, and FIG. 1H shows the similarly calculated local distribution of the radiation intensity of the protein sample which was labeled with isotopes of the I-131 type, the local distribution in FIG. 1F having being used for the calculation in both cases as the second local distribution which has been found.

FIG. 1I shows the difference of the local distributions in FIG. 1G and FIG. 1H, the protein sample which was labeled with isotopes of the I-125 type being represented in blue and the protein sample which was labeled with isotopes of the I-131 type being represented in orange. Black means that there is an equal number of the two isotopes.

The results of this embodiment are compatible with the results of embodiment 1, which can be used as reference results owing to the almost complete suppression of 1-131. The method according to the invention therefore provides reliable measurement results even if one radiation type is not fully suppressed when the second local distribution is found.

### THIRD EMBODIMENT

An absorber is used in order to obtain the corresponding modification factors in this embodiment of the invention, that is to say in order to carry out differential measurements of the samples containing the two isotopes I-125 and I-131. Similarly as in the first embodiment of the invention, a two-dimensional gel which contains proteins labeled with the two isotopes I-125 and I-131 is prepared. The phosphor imager IP is exposed to the local distribution of the radiation intensity of the 2D gel for a predetermined time. The phosphor imager IP is subsequently read using the phosphor imager readout device. The radiation intensity over the surface of the 2D gel is imaged in a matrix, with the values of the individual elements of the matrix, i.e. the pixels, representing the radiation intensity of the allocated region on the phosphor imager IP. This provides a matrix or image whose signal components are due both to I-125 and to I-131.

In order to separate the signal components, it is necessary to modify or reduce the intensity of one radiation type by suitable means and to find a second image or second local distribution.

By introducing an absorber of known thickness between the sample and the phosphor imager IP, the radiation intensity can be reduced selectively for each radiation type with different modification factors during the irradiation. Such an absorber may consist of metal, metal alloy, plastic, polymethyl methacrylate, polytetrafluoroethylene and/or another suitable material. In order to fulfill the function according to the invention, the absorber must absorb the radiation type (in this embodiment, photons from I-125 and beta particles from I-131) emitted by the radiation sources, which generates the signal on the detector, with respectively different modification factors.

In this embodiment, the intensity of the signal coming from I-131 can be reduced significantly relative to the signal from I-125 with the aid of the absorber. Absorbers made of elements with a low atomic number are more effective for this purpose (example: aluminum foil, thin polymethyl methacrylate etc.). The absorber thickness plays a key role in the absorption efficiency.

The first and the at least second local distributions of the radiation intensity may be found simultaneously with the aid of a “sandwich” arrangement comprising a phosphor imager IP, gel, absorber and phosphor imager IP. The images could alternatively be taken in succession, with or without an absorber, and this is preferred in the case of the invention. Of course, other radioactive isotopes could also be used for the invention disclosed here.

### FOURTH EMBODIMENT

In a fourth embodiment of the invention, as in the third embodiment, an absorber is used in order to modify the radiation intensity of the various radiation types differently.

Two samples of bovine serum albumin are labeled in this embodiment, one with I-125 and the other with I-131. Each of the samples is diluted four times in stages of 10. This provides 5 dilution stages of each sample, from undiluted to diluted by 1/10000. The radiation intensities consequently differ by a factor of 10,000. Each dilution stage of the first sample is mixed with each dilution stage of the second sample. This provides 25 samples with different mixing ratios, a 1 μl volume of each of them being applied to a square filter plate with a side length of 3 mm. The filter papers are arranged in a matrix arrangement on a flat support. The concentration of 1-125 decreases from bottom to top. The concentration of 1-131 decreases from left to right.

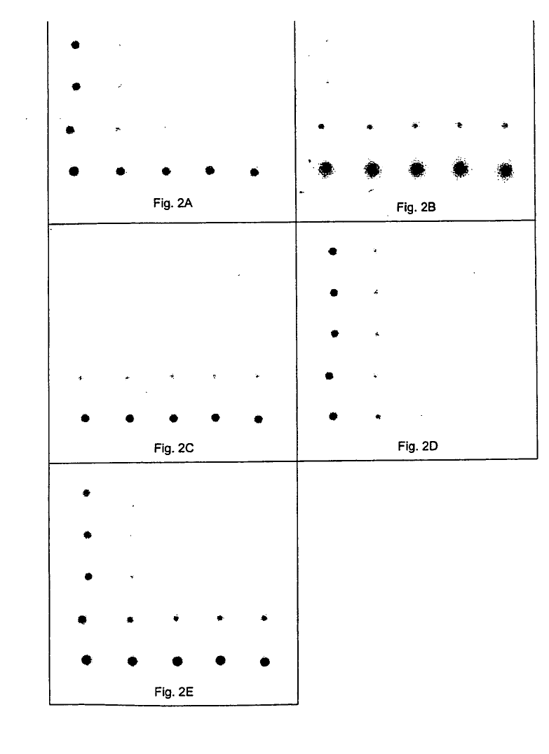

A phosphor imager IP is exposed to the radiation from the flat support for 24 hours. The phosphor imager IP integrates the radiation intensities from the two radiation sources of the samples. The local distribution of the stored radiation energy of the phosphor imager IP is subsequently read by a reader and formed into a pixel matrix or image, which is shown in FIG. 2A. The local distribution or pixel matrix found in this way contains components which come from both I-125 and I-131. Immediately after having found this first local distribution, a second local distribution is found, with an absorber being arranged between the flat support and the phosphor imager IP. This second local distribution is shown in FIG. 2B. The absorber consists of a 900 μm thick plastic sheet. The radiation intensities of the samples arranged on the flat support were integrated, or added up, using the same regions for both local distributions that were found. On the basis of these integrated radiation intensities, the local distribution of the individual isotopes is calculated separately with the aid of the method according to the invention from the two local distributions that have been found. FIG. 2C shows the calculated local distribution of the radiation intensity or isotope amount of the protein samples which were labeled with isotopes of the I-125 type, and FIG. 2D shows the calculated local distribution of the radiation intensity of the protein samples which were labeled with isotopes of the I-131 type. FIG. 2E shows the difference of the local distributions in FIG. 2C and FIG. 2D, the protein samples which were labeled with isotopes of the I-125 type being represented in blue and the protein samples which were labeled with isotopes of the I-131 type being represented in orange. Black means that there is an equal number of the two isotopes.

The undiluted sample labeled with I-125 at the bottom right and the undiluted sample labeled with I-131 at the top left were used to calculate the decay constants, which are needed in order to calculate the local distributions.

The calculated result corresponds within narrow error limits to the theoretically expected results. This shows that the method according to the invention works even with modification factors that are significantly more than 0%.

Possible Variants of the Embodiments

In the above embodiments of the invention, analysate molecules (proteins) are labeled with chemically identical radioisotopes (radioactive iodine). This method is extremely useful for carrying out differential analyses, because the differences in the intensity distribution of the radiation between the two samples are due to differences between the samples themselves. This also applies, for example, to proteins labeled with P-33 and P-32.

Proteins labeled with a combination of C-14, S-35 or P-33 produce different 2D PAGE images because carbon, sulfur and phosphate respectively occur with a different chemistry in the proteins. For instance, some proteins do not have any cysteine or methionine and therefore do not carry any sulfur, although they can be phosphorylated by a kinase so that they efficiently carry phosphate. Other proteins may have many amino acids containing sulfur, but might not phosphorylate. Nevertheless, the present invention is also suitable for extracting the synthetic images which correspond to the distribution of both isotopes when a combination of these isotopes is used according to the invention. The invention can in fact be applied to the differential measurement of any radioisotopes distributed over a surface or a similar area, even those with different surface structures, if they are suitable for phosphor imager analysis.

The radioactive isotopes suitable for the invention exhibit significant differences in the decay rate, the energy of emitted particles, the type of particles emitted, or a combination of these. A high isotope purity is desirable but not necessary.

A high chemical purity of the radioisotopes is desirable but not necessary. It is possible to apply the invention to samples which are labeled with different mixtures of isotopes, for example labeling one sample with 95% I-131, 5% I-125 and labeling the other sample with 5% I-131, 95% I-125. The labeling mixture may of course be very complex without departing from the concept or scope of the invention, and could for example contain trace amounts of many isotopes some of which are not covalently incorporated into the analysate molecules, such as K-40.

The present invention is independent of the method by which radioactive isotopes are incorporated into the analysate molecules. For example, proteins may be labeled by metabolic take-up, by post-harvest radio-iodination, by alkylation with radioactive reagents (of any chemistry) or by other methods.

Another application of phosphor imagers is the measurement of DNA and nucleic acid arrays. In such applications, for instance the frequency analysis of mRNA, it is possible for P-33, P-32, S-35, C-14, H-3 and other isotopes of any conventional type to be incorporated into hybridization molecules. A phosphor imager can be used according to the principles of the invention in order to carry out two-color analyses of these systems. Owing to the simple chemistry of DNA-like polymers, the radiation intensities of hybridization molecules that contain these radioisotopes may all be identical if the same sample is independently labeled and measured for each isotope. The principles described above also apply, of course, to protein arrays or any other arrays or semi-planar distributions of analysate molecules which are suitable for phosphor imager analysis.

It is apparent that applications of the invention may be implemented in the field of medical imaging, where two or more differential images are produced in order to extract synthetic images from two or more signal sources. One example might relate to the detection of certain radioactive trace elements combined with X-raying the body by using suitable absorbers according to the invention, and could be of great advantage in the field of medicine.

Clearly, the present invention is not restricted to phosphor imager IPs and radioactive measurements. The invention can also be applied to cases in which visible light rather than radioactivity is measured repeatedly, and the two signal components are extracted algorithmically. For example, an enzyme reaction may produce light of a similar wavelength to that which is emitted by a fluorophoric or luminescent molecule. By finding the intensity distribution of the light which is emitted together by the chemical reaction and the fluorophore, and the intensity distribution of the light which is emitted by the fluorophore, it is possible to determine the contributions of the light produced fluorescently and enzymatically in the original image since the enzymatic reaction produces a reduced amount of light. In this implementation, it is important for the reduction in the intensity of both light sources to be known or accurately calibratable.

All publications and patent applications cited in this disclosure are included by reference as part of the disclosure of the invention.

Terminology, Material and Methods

The terminology employed will be explained below, and a summary of the conventional materials and methods will be given.

An image is represented by a matrix, with the matrix cell values corresponding for example to the intensity of a signal which is recorded by a detector at a corresponding position on the imaged surface. The images may be stored in the conventional file formats for images.

Multicolor analysis is intended to mean that a plurality of samples are each labeled separately, mixed and separated together. The evaluation is then carried out individually for each sample, unaffected by the existence of the other respective samples. There are various methods for producing multicolour equations.

Genomics means the quantitative study of nucleic acids or polymers similar to nucleic acids.

Proteomics has been defined as the study of all proteins which are expressed in the genomes of the cells being studied. The acronym PROTEOME means PROTEins which are expressed by a genOME.

Wasinger V C, Cordwell S J, Cerpa-Poljak A, Yan J X, Gooley A A, Wilkins M R, Duncan W M, Harris R, Williams K L, Humphrey-Smith I. (1995) Progress with gene-product mapping of the Mollicutes: Mycoplasma genitalium. Electrophoresis, 16, 1090-4.

Here, proteomics means especially the study of molecules in which at least a part has been translated into a ribosome (a protein), as is known to persons skilled in the art, or a group of such proteins. Proteomics also covers the study of post-translational protein modifications, protein synthesis and breakdown rates, protein breakdown products and combinations of these.

Here the molecular profile, or profile, of a biological system such as a cell, a group of cells, a tissue or an organism, refers to a pattern of changes in the gene expression or protein expression or lipid composition or metabolite production of small molecules or temperature or metabolite secretion of small molecules, changes in sugar frequency or types, or the changes in post-translational protein modifications, or changes in the protein proteolysis, or changes in the ion secretion by one or more cell compartments, or in the ion take-up by one or more cell compartments between two or more biological systems being studied. Put more generally, the molecular profile of a biological system is a measure of the atoms which form this system, and in particular their chemical, spatial and temporal interrelationships.

Array means an ordered placement or arrangement. The term is used here in order to denote an ordered placement of oligonucleotides (including RNA, cDNA and genomic DNA) or of ligands for analysate molecules, such as affinity reagents for proteins, lipids and sugars or molecules which contain such functional groups (for example, a glycoprotein contains at least one sugar group or, for example, a lipoprotein contains at least one lipid group). The ordered molecules are positioned on a surface, for example a chip, and are used in order to capture complementary oligonucleotides (including RNA, cDNA and genomic DNA) or substrates for the ligands. Since the oligonucleotide or the ligand at any position in the arrangement is known, the sequence (of a nucleic acid) or a physical property (of a protein) can be determined by the position where the nucleic acid or the substrate bind to the array.

Protein Separation Methods

The application of electrophoresis for preparative purposes is an established technique, and there are various types of electrophoresis devices for preparative and analytical purposes. These devices and their associated principles can be divided into three categories.

Andrews A T. (1986) Electrophoresis: Theory, Techniques, and Biochemical and Clinical Applications. Oxford University Press.

Westermeier R. (1997) Electrophoresis in Practice: A Guide to Methods and Applications of DNA and Protein Separations. John Wiley and Sons, Weinheim.
a) zone electrophoresis
b) isotachophoresis
c) isoelectric focusing

Isotachophoresis uses hydrophilic matrices and typically exhibits a high resolution but low loading capacities. In combination with free electrophoresis, the method can be employed for micropreparative purposes.

Weber G, Bocek P. (1998) Stability of continuous flow electrophoresis. Electrophoresis, 19, 3094-3095.

Isoelectric focusing (IEF) is carried out either in liquid density gradients, in gel gradients or in multichamber devices, with a separation gel medium based on IEF immobilin.

Righetti P G. (1990) Immobilized pH gradients: theory and methodology. In: Laboratory techniques in biochemistry and molecular biology. Vol. 20 (editors R H Burdon, P H Knippenberg), page 397. Elsevier, Amsterdam.

Righetti P G, Bossi A, Wenisch E, Orsini G. (1997) Protein purification in multicomponent electrolyzers with isoelectric membranes. J. Chromatogr. B Biomed. Sci. Appl., 699, 105-15.

Zone electrophoresis comprises SDS PAGE (sodium dodecylsulfate polyacrylamide gel electrophoresis) protein gel systems buffered with glycine, bicine and tricine, which are conventionally used in protein analysis and are discussed in the literature citations given above.

Current proteomic techniques conventionally comprise the use of 2D PAGE (two-dimensional polyacrylamide gel electrophoresis), so that proteins are initially separated in the first dimension by differences in the p1 using IEF. These proteins are then subsequently separated in the second direction by molecular weight in an SDS PAGE gel.

Although 2D PAGE is currently the method which offers the greatest separation performance for biochemical purifications, the number of proteins and their modified isoforms which occur in eukaryotic cells, tissues and organisms exceeds the separation capacity of the best 2D PAGE systems. The frequency of various proteins and protein isoforms in complex eukaryotic cells, tissues and organisms also varies to a very great extent. The most frequent proteins in a eukaryotic cell may occur with more than 108 copies per cell. Examples of frequent proteins comprise particular cytoskeletal proteins or albumin in the liver and blood serum. Some proteins can exert significant biological effects when they are present in very small amounts, sometimes with only one pair of molecules per cell. Although the smallest amount with which proteins can exert biological activity is scarcely understood, this category may comprise examples such as telomerases and DNA polymerases in the progression from precancerous to cancerous cells. Other cases in which molecules with a low frequency in the protein mixtures are biologically relevant occur in the blood, where one pair of cytokine molecules per liter can be significant, or bone marrow where only one metastatic cancer cell among trillions of other cells can have fatal biological consequences for the patient. For a discussion of the number of proteins and protein isoforms which are to be expected in eukaryotic cells, and some implications for 2D PAGE in proteomics, see the Vuong et al.

Vuong G L, Weiss S M, Kammer W, Priemer M, Vingron M, Nordheim A, Cahill M A. Improved sensitivity proteomics by post harvest alkylation and radioactive labelling of proteins. 2000, Electrophoresis 21: 2594-2605.

The reproducibility of 2D PAGE is also notoriously low, so that several replicates of each gel are typically necessary in order to compile an average approximation of the composition of the typical 2D PAGE pattern of the system being studied with software packages. Some software packages are available on the market. One important application of proteomic studies involves determining the difference in the frequency of particular proteins or protein isoforms between two or more regimes of experimental interest, for example the frequency of proteins from control cells, tissues or organisms, in comparison with the corresponding frequencies from pharmacologically treated cells, tissues or organisms.

A range of other methods are available for protein separation which, without implying any limitation, comprise high-pressure liquid chromatography, thin layer chromatography, FPLC, gel filtration chromatography, ion exchange chromatography, microfluid systems such as capillary electrophoresis and microfluidics supported on chips, affinity reagents with microaffinity arrays and electrophoresis. The following literature references describe the prior art of separation methods which are used in proteomic research.

Daniel C. Liebler. Introduction to Proteomics: Tools for the New Biology. 300 pages 1^{st} edition (November 2001) Humana Press; ISBN: 0896039927.

Timothy Palzkill. Proteomics. 1^{st} edition (15 Nov. 2001) Kluwer Academic Publishers; ISBN 0792375653.

M J Dunn (editor) Proteomics Reviews 2001. (April 2001) VCH Verlags-ges. mbH; ISBN 3527303146.

Andre Schrattenholz (editor). Methods of Proteomie Research. Molecular analysis of protein expression [in German]. 2001. Spektrum Academische Verlag, Heidelberg. ISBN: 382741153X.

Issaq H J. The role of separation science in proteomics research. Electrophoresis 2001, 22:3629-3638.

Figeys D, Pinto D. Proteomics on a chip: promising developments. Electrophoresis 2001, 22:208-16.

Blagoev B, Pandey A. Microarrays go live new prospects for proteomics. Trends Biochem. Sci. 2001, 26:639-641.

Cahill D J. Protein and antibody arrays and their medical applications. J. Immunol Methods 2001, 250:81-91.

Paweletz C P, Charboneau L, Bichsel V E, Simone N L, Chen T, Gillespie J W, Emmert-Buck M R, Roth M J, Petricoin III E F, Liotta L a. Reverse phase protein microarrays which capture disease progression show activation of pro-survival pathways at the cancer invasion front. Oncogene 2001, 20:1981-1989.

Figeys D, Pinto D. Proteomics on a chip: promising developments. Electrophoresis 2001, 22:208-16.

Miklos G L, Maleszka R. Protein functions and biological contexts. Proteomics 2000, 1:169-178.

Lodish H, Berk A, Zipursky L S, Matsudaira P, Baltimore D, Darnell J. Molecular Cell Biology, 4^{th} edition, W.H. Freeman, New York, ISBN: 0-7167-3136-31986.

Patterson S D. Proteomics: the industrialization of protein chemistry. Curr. Opin. Biotechnol. 2000 11:413-418.

Protein Detection in Proteomics

Proteins can be detected by a multiplicity of methods which are described in the above literature references and which, without implying any limitation, comprise:
1) The bonding of dyes or colorants such as amide black, sulforhodamine B, agalma black or various silver coloring methods (above literature citations).

2) Spectroscopic detection of aromatic amino acids or peptide bonds.

3) Noncovalent association of fluorescent reagents.

4) Covalent bonding of fluorescent reagents, such as monobromium bimanthiolyte, naphthalene-2,7-disulfonic acid, fluorescene, Cy dye derivatives or a range of other reagents which are known to the persons skilled in the art. Fluorescent groups can be incorporated into protein molecules, for example, by alkylation of amino groups such as lysine or amino terminal groups, by alkylation of cysteine groups or by other methods.
5) Metabolic incorporation of radioactive isotopes, including H-3, C-14, S-35, P-32 and P-33, followed by radioactive detection.
6) Post-harvest radioactive labeling, such as protein iodination (Vuong et al., see above and citations given there), followed by radioactive detection.
7) Immunodetection in which antibodies or recombinantly produced affinity molecules interact specifically with proteins of interest, according to a range of standard protocols as disclosed by Harlow and Lane, Antibodies, A Laboratory Manual (Cold Spring Harbour Laboratory, Cold Spring Harbour, N.Y., 1988) or as mentioned in Hudson and Souriau, Expert Opin. Biol. Ther. 1:845-855 (2001) and in Manoutcharian et al., Curr. Pharm. Biotechnol. 2:217-233 (2001). Antibodies can be radiolabeled with or covalently bound to radioactive molecules, which allows them to be detected by radioactive detectors as described above in Manoutcharian et al. 8) Tagging is another way of detecting and determining a change in the protein expression. For example, the gene which encodes for the protein may be engineered in order to produce a hybrid protein which contains a detectable tag, so that the protein can be specifically detected by recognizing the tag. Systems are available which allow direct imaging and quantitative analysis of radioactive tagging, for example in gels on which proteins have been separated. Differences in the expression can be determined by recognizing differences in the amount of tagging present in test and control samples.

In cases when sample material is extremely limited, such as a certain biopsy or asservation methods for obtaining tissue from patients in clinical situations, or when the study should comprise detection of the analysate molecules with the lowest frequency, the most sensitive detection methods are extremely desirable. Among the aforementioned methods, the detection of radioisotope-labeled proteins by suitable detectors is several orders of magnitude more sensitive than any other method, and is therefore the method of choice.

Image Analysis Software

There are some elaborate software packages for the processing of graphical or digital data which are obtained from biological studies, such as protein gel analysis or nucleic acid array analysis. Examples for proteome analysis comprise the products Phoretix 2C from NonLinear Dynamics (Newcastle upon Tyne, UK), Delta 2D from Decodon GmbH (Greifswald, Germany), 3Z from Compugen (Tel-Aviv, Israel), Gellab II+ from Scanalytics (Faifax, Va.), Biolmage from Genomic Solutions (Ann Arbor, Mich.), Melanie 3 from GeneBio (Geneva), the Keppler program from Large Scale Biology Corporation (Vacaville, Ca), the Java enabled browser of the CAROL program (Freie Universitat, Berlin), the Flicker 2D gel image comparator (Lemkin P F. Comparing two-dimensional electrophoretic gel images across the Internet. Electrophoresis, 18:461-470 (1997) and image analysis products which are marketed by Amersham Biosciences (Freiburg, Germany).

Protein Identification in Proteomics

Mass spectrometry is gaining increased importance in proteomics, not only for the identification of proteins which have been separated by 2D PAGE but also for the direct detection and relative quantification of proteins independently of 2D PAGE. Other methods for protein identification comprise Edman breakdown, amino acid analysis and other methods which are known to the persons skilled in the art. Many of these methods involve the acquisition of data from the protein being studied and comparison of these parameters with a list of parameters which are predicted by theoretical analysis of nucleic acid and protein databases.

Anderson N L, Matheson A D, Steiner S. Proteomics: applications in basic and applied biology. Curr. Opin. Biotechnol. 2000, 11:408-412.

Gygi S P, Aebersold R. Mass spectrometry and proteomics. Curr. Opin. Chem. Biol. 2000, 4:489-494.

Lee K H. Proteomics: a technology-driven and technology-limited discovery science. Trends Biotechnol. 2001 June; 19(6):217-222.

Patterson S D. Proteomics: the industrialization of protein chemistry. Curr. Opin. Biotechnol. 2000 11:413-418.

Godovac-Zimmermann J, Brown L R. Perspectives for mass spectrometry and functional proteomics. Mass Spectrom. Rev. 2001, 2001, 20:1-57.

P. James (editor) Protein Research: Mass Spectrometry (Principles and Practice) 235 pages (December 2000) Springer Verlag; ISBN: 3540672567.

Genomics

A range of methods for detecting and comparing the extent of gene expression in cells, organisms or viruses or other systems with transcription activity is known in the prior art.

One standard method for such comparisons is the Northern blot. In this technique, RNA is extracted from the sample and applied to a range of gels suitable for the RNA analysis, which are then carried out in order to separate the RNA according to size.

Sambrook J. et al., 1989. Molecular Cloning, A Laboratory Manual. Cold Spring Harbour Laboratory Press, Cold Spring Harbour, N.Y. 2^{nd} edition.

The gels are then blotted (as described above in Sambrook) and hybridized onto samples for RNAs of interest. The samples may be radioactive or non-radioactive. For example, hybridization with the sample may be observed and analyzed by chemiluminescent detection of the bound samples using the Genius System (Boehringer Mannheim Corporation, Mannheim, Germany) according to the manufacturer's data. Equal loading of the RNA in the tracks may, for example, be evaluated by dying the ribosomal RNA bands with ethidium bromide. As an alternative, the samples may be radiolabelled and autoradiographically detected with a sample for a gene and a photographic film or a phosphor imager or according to the present invention with samples for more than one gene.

The RNA can be amplified by a range of methods and then detected. For example, Marshall, U.S. Pat. No. 5,686,272 discloses the amplification of RNA sequences with a ligase chain reaction LCR. LCR is described by Landegren et al., Science, 241: 1077-1080 (1998); Wu et al., Genomics, 4:560-569 (1989); Barany, in PCR Methods and Application, 1:5-16 (1991); and Barany, Proc. Natl. Acad. Sci. USA, 88:189-193 (1991). The RNA can also be converted by reverse transcription into complementary DNA (cDNA) and then amplified by LCR, polymerase chain reaction (PCR) or other methods. An example of a method for carrying out the reverse transcription of RNA is disclosed in U.S. Pat. No. 5,705,365. A selection of suitable primers and PCR protocols is disclosed, for example, in Innis M. et al., ed. PCR Protocols 1990 (Academic Press, San Diego Calif.). Differential expression of messenger RNA (mRNA) can also be compared by reverse transcription of mRNA into cDNA, which is then cleaved by restriction enzymes and electrophoretically separated, so that it is possible to compare the cDNA fragments, as disclosed in Belavsky, U.S. Pat. No. 5,814,445.

Typically, primers at the 5′ end are labeled with biotin or one of the many fluorescent dyes. Samples are conventionally labeled with an enzyme such as horseradish peroxidase (HRP) and alkaline phosphatase, see Levenson and Chang, Nonisotropically Labeled Probes and Primers in: Innis et al., although they may also be labeled with a psoralen-biotin, for example. Detailed protocols for labeling primers and for synthesizing enzyme-labeled samples are specified in Levenson and Chang. The samples may also be labeled with radioactive isotopes. An exemplary protocol for synthesizing radioactively labeled DNA and RNA samples is given in Sambrook. A number of reagents are available for the incorporation of various radionuclides into DNA samples, including enzyme precursor substrates with 3H, 14C, 32P, 35S and other elements which are not present in naturally occurring nucleic acids, for example radioactive iodine. It is also possible to produce primers which contain a large range of other radioactive elements that are covalently bound to the primer, as is known to the persons skilled in the art. A range of methods for the detection of PCR products are known. They generally contain a step which allows hybridization of the sample and of the PCR product, which is followed by one or more development steps in order to facilitate detection.

When a radioactively labeled sample is to be used, PCR products with which the sample is hybridized may be detected by autoradiography. For example, biotinylated dUTP (Bethesda Research Laboratories, MD) may be used for the amplification. The labeled PCR products can then be separated on an agarose, applied to a nylon filter by Southern Transfer and, for example, detected by a detector system with streptavidin/alkaline phosphatase. A protocol for the detection of biotinylated dUTP which has been incorporated is described, for example, in Lo et al., Incorporation of Biotinylated dUTP, in Innis et al. Lastly, the PCR products may be put on agarose gels, and nucleic acids may be detected by a dye such as ethidium bromide, which specifically recognizes nucleic acids.

Sutcliffe, U.S. Pat. No. 5,807,680 discloses a method for the simultaneous identification of differentially expressed mRNAs and measurement of relative concentrations. The technique, which comprises the formation of cDNA with anchor primers followed by PCR, makes it possible for virtually all mRNA which is expressed in a tissue to be visualized as a clear band on a gel, the intensity of which corresponds approximately to the mRNA concentration.

Another group of techniques uses analysis of the relative transcription expression values. Four such approaches have recently been developed in order to allow comprehensive analysis with a high throughput. First, cDNA can be obtained by reverse transcription from RNA in the sample (as described in the citations above) and subjected to simple sequencing of the 5′ and 3′ ends, in order to define expressed sequence tags (ESTs) for the genes, which are expressed in the test, and control samples. Counting the relative representation of the tags of different samples gives an estimate of the relative representation of the gene transcript in the samples.

Secondly, a variation of ESTs known as SAGE (serial analysis of gene expression) has been disclosed, which allows quantitative and simultaneous analysis of a large number of transcripts. The technique uses the isolation of short diagnostic sequence tags and sequencing in order to display patterns of the gene expression characteristics of a target function, and have been used in order to compare expression values of, for example, thousands of genes in normal and tumor cells. See Velculescu at al., Science 270:368-369 (1995), Zhang at al., Science 276:1268-1272 (1997).

Thirdly, approaches based on differential display have been developed. In these approaches, fragments defined by specific sequence restrictors can be used as individual identifiers for genes when they are coupled with information about the fragment length in the expressed gene. The relative representation of an expressed gene in a cell can then be estimated by the relative representation of the fragment assigned to the gene. Examples of some approaches are restriction enzyme analysis of differentially expressed sequences (READS) used by Gene Logic Inc., and the total gene expression analysis from Digital Gene Technologies Inc. CLONETECH, Inc. (Palo Alto, Calif.) market the Delta® differential display kit for identifying differentially expressed genes by PCR. Another method is the GeneCalling system from CuraGen Corp., New Haven Conn., which combines gene identification with database query of a restriction endonuclease fingerprint, confirmed by comparative PCR by using gene-specific oligonucleotides, so as to optimize gene isolation procedures.

Fourthly, in preferred embodiments, the detection is carried out by a range of techniques for hybridization analysis. In these approaches, RNA from the sample of interest is subjected to a reverse transcription in order to obtain labeled cDNA. The cDNA is then hybridized, typically with oligonucleotides or cDNAs with a known sequence, which are arranged in a known sequence on a chip or other area. The position of the oligonucleotide which is hybridized with the labeled cDNA gives sequence information about the cDNA, while the amount of labeled RNA or cDNA gives an estimate of the amount of the relative RNA or cDNA in the original sample. The technique furthermore allows simultaneous hybridization with two or more different detectable labels, such as two or more radioactive or fluorescent labels, according to claim 1 of the present invention. The hybridization results give a direct comparison of the relative expression of the samples. The prior art is reviewed by King and Sinha, JAMA 286:2280-2208 (2001) and Jain, Science, 294:621-623 (2001) and the literature citations in these.

A range of kits for hybridization analysis are available on the market. These kits make it possible to identify specific RNA or cDNA molecules on high-density formats, including filters, microscope slides, microchips and mass spectrometry techniques. For example, Affymetrix Inc. (Santa Clara, Calif.) markets GeneChip® sample arrays which contain thousands of different oligonucleotide samples with known sequences, lengths and locations in the array for high-precision sequencing of genes. Synthetic oligonucleotide arrays combined with PCR methods can detect transcription conditions with one copy per cell in complex biological samples, and an inkjet oligonucleotide array technique can be used for accurate application. See Hughes, T. R. et al., Nature Biotechnology, 19:342-347 (2001). CLONTECH's Atlas® cDNA expression array makes it possible to measure the expression patterns of 588 selected genes. The Gene Discovery Module from Hyseq Inc. (Sunnyvale, Calif.) allows high throughput RNA screening without prior sequence information, with a sensitivity of 1 mRNA copy per cell. Incyte Pharmaceuticals Inc. (Palo Alto, Calif.) offers microarrays which, for example, contain ordered oligonucleotides of human cancer and signal transduction genes. Technologies used by other companies are discussed, for example, in Service, R.; Science 282:396-399 (1998) and Clarke P. A. Biochem. Pharmacol., 62:1311-1136 and the citations in these.

Measurement of Radioactive Radiation

The first electrical devices developed for radiation detection were ionization detectors. These instruments are based on the direct capture of electrons and ions which are produced in a gas by radiation. There are two basic types of detectors, which are referred to as ionization chambers and proportional counters.

Another detection device is the scintillation detector. It is based on the fact that when a nuclear particle or radiation strikes them, certain materials emit one or more photons, that is to say a scintillation. By coupling to an amplifier device such as a photomultiplier, these scintillations can be converted into electrical pulses which can then be analyzed and electronically counted in order to provide information about the incident radiation.

Semiconductor detectors are also widely used detection devices which are based on crystalline semiconductor materials, primarily silicon and germanium. These detectors are referred to as solid-state detectors. The functional principle of semiconductors is similar to that of gas ionization devices. Instead of a gas, the medium is a solid semiconductor material. The advantage of semiconductor detectors in comparison with ionization chambers is their high energy resolution.

The following textbook describes the principles of radioactive detection methods:

Knoll, G. F.: Radiation detection and measurements, 802 pages, 3^{rd} edition, December 1999, John Wiley and Sons, ISBN 0471073385.

1. A method for determining the local distribution of at least two sets of point radiation objects each with a set-specific radiation type on a common support surface, having the following steps:
a) finding a first local distribution of the radiation intensity of the support surface,
b) modifying the intensity of at least one set-specific radiation type with an associated modification factor,
c) finding at least a second local distribution of the radiation intensity of the support surface, and
d) calculating the local distributions of each of the at least two sets of point radiation objects individually from the first and the at least second local distributions which have been found.

2. The method as claimed in claim 1, characterized in that the modification step b) and the step c) of finding the at least second local distribution of the radiation intensity of the support surface are repeated at least once, preferably with a different modification factor.

3. The method as claimed in claim 1 or 2, characterized in that a local distribution of the radiation intensity which has been found is represented by an associated pixel matrix, with a pixel value of the matrix representing the radiation intensity of an associated position on the support surface.

4. The method as claimed in claim 3, characterized in that the local distribution of each of the at least two sets of point radiation objects is determined individually on the basis of the pixel matrices.

5. The method as claimed in claim 3 or 4, characterized in that the local distribution of each of the at least two sets of point radiation objects is determined individually on the basis of intensities which are obtained by adding up pixel values of defined, in particular neighboring, elements of the pixel matrix.

6. The method as claimed in one of the preceding claims, characterized in that each set of point radiation objects consists of at least one radiating, in particular radioactively radiating, type of isotope.

7. The method as claimed in one of the preceding claims, characterized in that each set of point radiation objects consists of light-emitting, in particular fluorescent, phosphorescent and/or luminescent, substances.

8. The method as claimed in one of the preceding claims, characterized in that there is at least one calibration point with known radiation for at least one set of different point radiation objects at at least at one position on the support surface.

9. The method as claimed in one of the preceding claims, characterized in that the ratio of the modification factors of the set-specific radiation types is between 5% and 90%, preferably between 10% and 70%, in particular between 15% and 50%.

10. The method as claimed in one of the preceding claims, characterized in that the modification factor is obtained by using at least one absorber.

11. The method as claimed in one of the preceding claims, characterized in that the modification factor is obtained by radioactive decay of the point radiation objects of the at least two sets, with the respective point radiation objects of the at least two sets preferably having different half-lives.

12. The method as claimed in one of the preceding claims, characterized in that the modification factor is obtained by a differing sensitivity of a detector, used for finding a local distribution of the radiation intensity of the support surface, for the respective set-specific radiation type of the at least two sets of point radiation objects.

13. The method as claimed in one of the preceding claims, characterized in that the local distributions are found by using a position-resolving detector for alpha, beta, gamma and/or X-radiation.

14. The method as claimed in one of the preceding claims, characterized in that the local distributions are found by using a so-called phosphor imager detector.

15. The method as claimed in claim 14, characterized in that a differing sensitivity of a phosphor imager detector, used for finding a local distribution of the radiation intensity of the support surface, for the respective set-specific radiation type is achieved by the phosphor of the phosphor imager detector additionally containing atoms with a high atomic number Z, in particular lead, and/or atoms with a low atomic number Z.

16. The method as claimed in claim 15, characterized in that the first local distribution of the radiation intensity of the support surface is found by using a first phosphor imager detector, and the at least second local distribution of the radiation intensity of the support surface is found simultaneously by using at least a second phosphor imager detector, the sensitivity of the first phosphor imager detector and the sensitivity of the at least second phosphor imager detector differing for the respective set-specific radiation type.

17. The method as claimed in claim 16, characterized in that the first and the at least second phosphor imager detectors are applied to opposite sides of the support surface.

18. The method as claimed in one of the preceding claims, characterized in that the local distributions are found by using a so-called flat panel detector.

19. The method as claimed in one of the preceding claims, characterized in that the first and the at least second local distributions of the radiation intensity are found by using the same type of detector.

20. The method as claimed in one of the preceding claims, characterized in that the at least two substances to be analyzed are respectively labeled with a set of point radiation objects, the substances labeled in this way are mixed and the mixture is subsequently applied to the support surface, particularly in the form of a flat analysis medium.

21. The method as claimed in claim 20, characterized in that the substances to be analyzed are peptides, proteins and/or oligonucleotides.

22. The method as claimed in claim 20 or 21, characterized in that the analysis medium is a protein gel, nucleic acid array, protein array, ELISA array and/or a blot.
